(19) United States
(12) Patent Application Publication
(10) Pub. No.: US 2025/0137960 A1
(43) Pub. Date: May 1, 2025

(54) MONITORING A DIELECTRIC CONSTANT OF CURING CONCRETE

(71) Applicant: Intellitronx Robotics and Data Analytics Corp., Starke, FL (US)

(72) Inventors: Dan G. Zollinger, Bryan, TX (US); Jesse Hubert Livesay, JR., Albuquerque, NM (US)

(21) Appl. No.: 18/929,151

(22) Filed: Oct. 28, 2024

Related U.S. Application Data
(60) Provisional application No. 63/546,148, filed on Oct. 27, 2023.

(51) Int. Cl. G01N 27/22; G01N 33/38

(52) U.S. Cl. CPC G01N 27/221; G01N 27/223; G01N 33/383

(57) ABSTRACT

A dielectric probe includes an electrically conductive element and a wire. The electrically conductive element introduces electricity into a material (e.g., curing concrete, etc.) whose dielectric constant is being evaluated or receives electricity from the material. When a pair of dielectric probes is used together, oriented vertically within a material at spaced-apart locations in the material, they may be used to determine a dielectric constant of a location or an area of a material at a particular point in time. The dielectric constant may be used to determine a water content of the material.

### CROSS-REFERENCE TO RELATED APPLICATION

A claim for priority to the Oct. 27, 2023 filing date of U.S. Provisional Patent Application No. 63/546,148, titled MONITORING HUMIDITY AND WATER CONTENT OF CONCRETE CURING (“the '148 Provisional Application”), is hereby made. The entire disclosure of the '148 Provisional Application is hereby incorporated by reference.

### TECHNICAL FIELD

This disclosure relates generally to apparatuses, systems, and methods for monitoring concrete as it cures. More specifically, this disclosure relates to dielectric probes that monitor the dielectric constant of curing concrete, which may be used to determine the water content of the curing concrete, and, optionally, a humidity of the curing concrete. This disclosure also relates to methods for monitoring the dielectric constant of curing concrete to determine the water content of the curing concrete and, optionally, the humidity of the curing concrete to determine how water is moving through the curing concrete.

### BACKGROUND

The curing of concrete can be a temperamental process. If curing conditions, including the temperature and water content of the concrete, are not correct during the early curing period, or for the first few days (e.g., three days, seven days, etc.) of the curing process, hydration of the concrete (i.e., the reaction between water and cement in the concrete) may be adversely affected, which may prevent the concrete from setting and hardening properly and, thus, negatively affect the durability of the concrete. Atmospheric changes (i.e., changes in the weather) may adversely affect hydration and, thus, the quality of the curing concrete. A variety of so-called failures can result from improper hydration, including cracking, spalling, curling, and loss of strength.

When concrete pavement is placed, evaporation happens at the surface, not uniformly throughout the pavement. When the concrete pavement is not properly hydrated, the surface may shrink laterally while lower, more hydrated portions of the concrete substantially retain or retain their lateral dimensions (e.g., they do not experience significant lateral shrinkage). Such asymmetric shrinkage may result in strain within the concrete pavement. One consequence of such strain may be a concrete pavement with a top that is not fully supported by its bottom, which may cause roughness and concave surfaces in sections of the concrete pavement.

Evaluations of the effectiveness of concrete curing are typically conducted in the laboratory using ASTM C 156 (Water Retention by Concrete Curing Materials). The deficiencies of evaluating concrete curing processes in this manner include: (1) test conditions hold little relevance to field conditions; (2) laboratory measurements are often not useful or transferable to the environments in which concrete is cured; and (3) they provide a questionable basis for moisture loss limits and have limited relevance to the short-term and long-term performance of the concrete.

Conventionally, a variety of techniques have been used to prevent failures from occurring as concrete cures. These include passive controls, such as the use of membranes or curing compounds over the surface of curing concrete, the inclusion of shrinkage additives or concrete reinforcing fibers in the concrete mixture, and saw cutting the concrete. The effectiveness of passive controls is still subject to atmospheric conditions (i.e., the weather) and changes in atmospheric conditions. Moreover, the use of passive controls does not provide information that may be useful in compensating for changes in atmospheric conditions or the effects such changes may have on hydration of the curing concrete.

Active controls have the potential to prevent failures from occurring as concrete cures by providing data during curing that may lead to real-time adjustments to the curing process. One example of an existing active control device is known as a concrete curing maturity meter. The concrete curing maturity meter measures the dry bulb temperature (T) and dew point temperature (Dₚ) of the concrete, from which relative humidity (RH) is calculated, as well as the temperature (e.g., dry bulb temperature, etc.), relative humidity, wind speed, and solar radiation of the environment in which the curing concrete is located. Dry bulb temperature is the temperature of air as measured by a thermometer that is not affected by moisture or radiation; dry bulb temperature is commonly referred to as “air temperature.” Dew point temperature is the temperature air needs to be cooled to (at constant pressure) to achieve a relative humidity of 100%. With the dry bulb temperature and dew point temperature, relative humidity may be calculated as follows:

$$RH = 100 \times \frac{{e}^{\frac{17.625 \times {D}_{p}}{243.04 + {D}_{p}}}}{{e}^{\frac{17.625 \times T}{243.04 + T}}}$$

The concrete curing maturity meter positions a pair of chilled mirror dew point temperature (DPT) sensors, or chilled mirror hygrometers, within the curing concrete to monitor its dry bulb temperature and dew point temperature. One of the chilled mirror hygrometers measures the dry bulb temperature and dew point temperature slightly below the surface of the curing concrete, while the other chilled mirror hygrometer provides the dry bulb temperature and dew point temperature from deeper within the curing concrete.

### SUMMARY

In various aspects of this disclosure, the water content and, optionally, the humidity of concrete are monitored as the concrete cures. Water is essential to concrete curing. Water reacts with cement in the concrete in a chemical reaction called “hydration” to form chemical bonds within the concrete to strengthen the concrete. Knowing the water content of curing concrete (e.g., volumetric fraction of water in the curing concrete, etc.) is important in determining whether the concrete is properly hydrated throughout the curing process.

The water also acts like a glue. It holds particles together through its surface tension, or suction. When the humidity of freshly placed concrete is 100%, that suction is zero because the concrete is totally saturated with water. As the water evaporates from the concrete over time, the water tends to hold everything increasingly tighter together. One of the reasons concrete is so strong is because of the effects of water as the concrete cures (i.e., its suction).

The water content of concrete or another material may be determined by measuring a dielectric constant (e.g., a dielectric constant) of the concrete or other material. The water content at one location within a material may differ from the water content at another location within the material. Accordingly, a probe of this disclosure may comprise a dielectric probe that may be used in determining the dielectric constant of a material at a particular location or in a particular area; and that data may be used to determine the volumetric water content of the location or area of the material (e.g., of curing concrete, etc.). The dielectric probe may comprise an electrical probe positioned so as to contact the material being monitored. In some embodiments, a dielectric probe of this disclosure may be configured to measure only the dielectric constant of the material. In other embodiments, the dielectric probe may be used to measure other properties of the material, including, without limitation, a temperature of the material, a humidity of the material, or the like.

Humidity provides an indicator of characteristics of the movement of water through the concrete. Water vapor and moisture in the concrete move in one direction-up, from the bottom of the concrete slab to the top of the concrete slab, with water leaving the slab by evaporating from the top surface of the concrete slab. Thus, the movement of water through the curing concrete can be referred to as a “one-dimensional transport process.” Monitoring the humidity of curing concrete may be helpful in identifying which areas of the curing concrete are drying more rapidly than others.

A dielectric probe of this disclosure may facilitate monitoring of both the water content of a particular location of a material, such as concrete (e.g., by measuring a dielectric constant of the material at the particular location, etc.) and the humidity of that location of the material. In various embodiments, such a probe may include a body with a tip that facilitates insertion of the body into the material (e.g., the tip may be rounded, tapered, pointed, etc.), an electrically conductive element that may be used to determine the dielectric constant of the material, and an optional humidity sensor that may facilitate measurement of a humidity of the material.

The body of the probe may be electrically conductive, which may enable the body of the probe to measure the dielectric constant of the material.

In embodiments where the probe may be used to measure properties other than the dielectric property (e.g., temperature, humidity, etc.), the body of the probe comprise a sample tube. The sample tube may comprise a sample tube of the type disclosed by U.S. patent application Ser. No. 17/722,295, titled MONITORING OF CURED CONCRETE (“the '295 Application”), the entire disclosure of which is hereby incorporated herein. In in such an embodiment, the body of the probe, i.e., the sample tube, may be used to measure the dielectric constant of the material and may collect one or more humidity samples, which may then be communicated to a relative humidity sensor that is remote from the body, and, thus, be part of a system such as that disclosed by the '295 Application. A plurality of sample tubes may communicate with a single relative humidity sensor. In some embodiments, the relative humidity sensor may comprise a chilled mirror hygrometer.

Alternatively, the body of the probe may include a passage, or one or more sampling chambers, each of which may carry a relative humidity sensor. Each relative humidity sensor carried by the body of the probe may comprise a chilled mirror hygrometer. In some embodiments, the body of such a probe may carry a pair of chilled mirror hygrometers, with a first chilled mirror hygrometer providing a first dry bulb temperature and a first dew point temperature at a first location along a length of the body and a second chilled mirror hygrometer providing a second dry bulb temperature and a second dew point temperature at a second location along the length of the body.

The humidity and moisture content data obtained with such a dielectric probe may be used to characterize a change in the measured strain of curing concrete.

A method for characterizing a change in measured strain of curing concrete may include introducing at least one dielectric probe into the curing concrete, measuring the relative humidity of the curing concrete and/or the dielectric constant of the curing concrete with the at least one probe, and determining the measured strain of the curing concrete as a function of the relative humidity and/or dielectric constant of the curing concrete. In some embodiments, the relative humidity and dielectric constant of the curing concrete may measured.

The at least one probe may be introduced through a surface of the curing concrete. Introduction of the probe may include forcing a tip of a body of the at least one probe into and through the surface of the curing concrete. In some embodiments, a plurality of probes may be introduced into the curing concrete at different locations, thereby facilitating measurement of the relative humidity and/or moisture content of the curing concrete at each location where a probe has been placed. The different locations may be spaced apart from each other substantially evenly over the surface of the curing concrete. The at least one probe may be introduced into fresh concrete, new concrete (e.g., concrete that has been in place for less than seven (7) days), including set concrete, or hardened concrete (e.g., concrete that has been in place for at least seven (7) days).

Information on the extent to which various locations of the curing concrete are hydrated and hydration/drying trends across the surface of the concrete may be used in determining how to treat the concrete. For example, such information may be used in determining where to further hydrate and/or apply curing aids to the concrete.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

### In the Drawings:

FIG. 1 is a side view of an embodiment of a dielectric probe that may be inserted vertically into a material whose dielectric constant is to be measured;

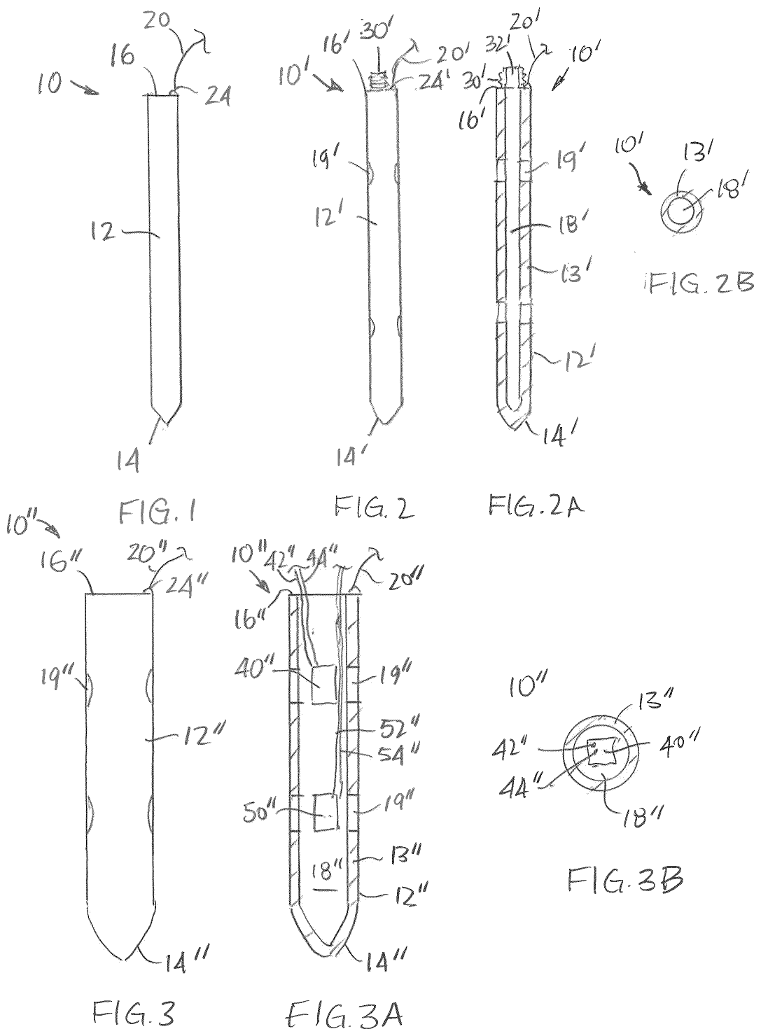

FIG. 2 is a side view of another embodiment of dielectric probe that may be inserted vertically into a material whose dielectric constant is to be measured;

FIG. 2A is a cross-sectional representation of the dielectric probe of FIG. 2, taken along a longitudinal axis of the dielectric probe;

FIG. 2B is a cross-sectional representation of the dielectric probe of FIG. 2, taken transverse to the longitudinal axis of the dielectric probe;

FIG. 3 is a side view of another embodiment of dielectric probe that may be inserted vertically into a material whose dielectric constant is to be measured;

FIG. 3A is a cross-sectional representation of the dielectric probe of FIG. 2, taken along a longitudinal axis of the dielectric probe;

FIG. 3B is a cross-sectional representation of the dielectric probe of FIG. 2, taken transverse to the longitudinal axis of the dielectric probe;

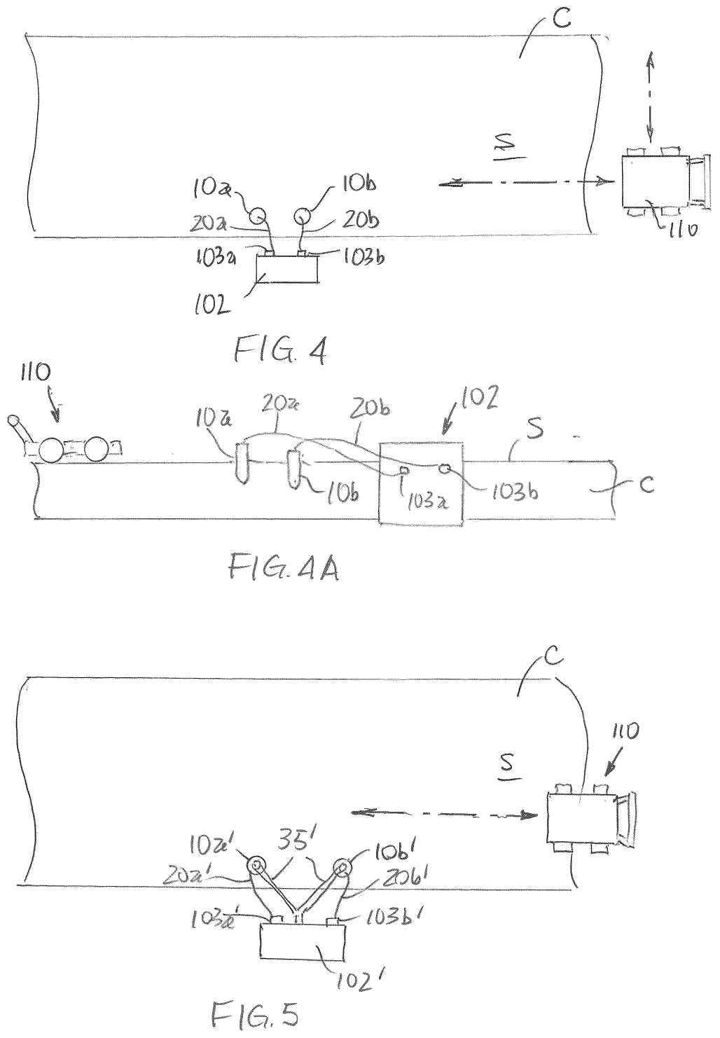

FIG. 4 is a schematic representation of an embodiment of a monitoring system, which may be used to monitor a dielectric constant of a material;

FIG. 4A is a cross-sectional representation of the material whose dielectric constant is monitored using the embodiment of the monitoring system depicted by FIG. 4; and

FIG. 5 is a cross-sectional representation of another embodiment of a monitoring system, which may be used to monitor a dielectric constant of a material and a humidity of the material.

### DETAILED DESCRIPTION

FIG. 1 depicts an embodiment of a dielectric probe 10 of this disclosure. The dielectric probe 10 comprises an electrically conductive element 12 and a wire 20.

The electrically conductive element 12 is elongated. The electrically conductive element may be substantially straight or straight. The electrically conductive element 12 is solid.

The electrically conductive element 12 may comprise, consist of, or consist essentially of an electrically conductive material. As an example, the electrically conductive material may be copper, aluminum, steel, or any other suitable electrically conductive material.

The electrically conductive element 12 may have any suitable dimensions. For example, the electrically conductive element 12 may have a diameter of about one-quarter inch (¼″) (about 0.635 cm), although smaller diameters (e.g., 0.1 inch (0.25 cm), 0.15 inch (0.4 cm), 0.2 inch (0.5 cm), etc.) and larger diameters (e.g., 0.3 inch (0.75 cm), 0.35 inch (0.9 cm), 0.4 inch (1 cm), etc.) are also within the scope of this disclosure. A length of the electrically conductive element 12 may enable it to be embedded vertically a desired distance within a material whose dielectric constant is to be measured. For example the electrically conductive element 12 may have a length of about three inches (about 7.5 cm) or more (e.g., 3 1/4 inches (8.25 cm), 3 1/2 inches (8.9 cm), 3 3/4 inches (9.5 cm), 4 inches (10.1 cm), 4 1/2 inches (11.4 cm), 5 inches (12.7 cm), 5 1/2 inches (14 cm), 6 inches (15.25 cm), etc.). In a specific embodiment, the electrically conductive element 12 may comprise a one-quarter inch (¼″) (0.635 cm) diameter copper rod with a length of about six inches (about 15.25 cm).

The electrically conductive element 12 has a first end 14 and a second end 16. During use of the dielectric probe 10, the first end 14 is inserted into a material, such as concrete (e.g., freshly poured, pliable concrete, set concrete, hardened concrete, etc.), whose dielectric constant is to be measured. The first end 14 may be oriented perpendicular to a longitudinal axis of the electrically conductive element 12. Alternatively, a configuration of the first end 14 may facilitate its insertion into the material whose dielectric constant is to be measured; for example, the first end 14 may be rounded, tapered to a point along the longitudinal axis of the electrically conductive element 12, tapered to define a wedge shape, etc.

When the dielectric probe 10 is used, the second end 16 of the electrically conductive element 12 remains outside of the material whose dielectric constant is to be measured. A first end 24 of the wire 20 may be electrically coupled to the second end 16 of the electrically conductive element 12. Electrical coupling between the first end 24 of the wire 20 of the dielectric probe 10 and the second end 14 of the electrically conductive element 12 may be achieved in any suitable manner. Without limitation, the first end 24 of the wire 20 may be soldered to the second end 16 of the electrically conductive element 12, a mechanical coupler may secure the first end 24 of the wire 20 to the second end 16 of the electrically conductive element 12 and establish electrical communication between them, etc.

FIGS. 2, 2A, and 2B depict another embodiment of a dielectric probe 10′ of this disclosure. The dielectric probe 10′ includes an electrically conductive element 12′ and a wire 20′.

The electrically conductive element 12′ is elongated. The electrically conductive element 12′ may be substantially straight or straight. The electrically conductive element 12′ is somewhat tubular, with a passage 18′ extending partially through its length. In addition, one or more apertures 19′ extend through one or more sides 13′ of the electrically conductive element 12′ to establish communication between an exterior of the electrically conductive element 12′ and the passage 18′ within the electrically conductive element 12′.

The electrically conductive element 12′ may comprise, consist of, or consist essentially of an electrically conductive material. As an example, the electrically conductive material may be copper, aluminum, steel, or any other suitable electrically conductive material.

The electrically conductive element 12′ may have any suitable dimensions. For example, the electrically conductive element 12′ may have a diameter of about one-quarter inch (¼″) (about 0.635 cm), although other diameters (e.g., 0.3 inch (0.75 cm), 0.375 inch (0.95 cm), 0.4 inch (1 cm), 0.5 inch (1.25 cm), 0.6 inch (1.5 cm), 0.625 inch (1.6 cm), etc.) are also within the scope of this disclosure. A length of the electrically conductive element 12′ may enable it to be embedded vertically a desired distance within a material whose dielectric constant is to be measured. For example, the electrically conductive element 12′ may have a length of about three inches (about 7.5 cm) or more (e.g., 31/4 inches (8.25 cm), 31/2 inches (8.9 cm), 33/4 inches (9.5 cm), 4 inches (10.1 cm), 41/2 inches (11.4 cm), 5 inches (12.7 cm), 51/2 inches (14 cm), 6 inches (15.25 cm), etc.). In a specific embodiment, the electrically conductive element 12′ may comprise a one-half inch (½″) (1.27 cm) diameter copper tube with a length of about six inches (about 15.25 cm).

The electrically conductive element 12′ has a first end 14′ and a second end 16′. During use of the dielectric probe 10′, the first end 14′ is inserted into a material, such as concrete, whose dielectric constant is to be measured. The first end 14′ may be closed; thus, the passage 18′ through the electrically conductive element 12′ may not open to the first end 14′. Such a configuration may prevent a material from entering the passage 18′ while the electrically conductive element 12′ is introduced into the material.

A configuration of the first end 14′ may facilitate its insertion into the material whose dielectric constant is to be measured; for example, the first end 14′ may be rounded, tapered to a point along the longitudinal axis of the electrically conductive element 12′, tapered to define a wedge shape, etc.

When the dielectric probe 10′ is used, the second end 16′ of the electrically conductive element 12′ remains outside of the material whose dielectric constant is to be measured.

A first end 24′ of the wire 20′ of the dielectric probe 10′ may be electrically coupled to the second end 16′ of the electrically conductive element 12′. Electrical coupling between the first end 24′ of the wire 20′ and the second end 14′ of the electrically conductive element 12′ may be achieved in any suitable manner. Without limitation, the first end 24′ of the wire 20′ may be soldered to the second end 16′ of the electrically conductive element 12′, a mechanical coupler may secure the first end 24′ of the wire 20′ to the second end 16′ of the electrically conductive element 12′ and establish electrical communication between them, etc.

The passage 18′ may open to the second end 16′ of the electrically conductive element 12′. The passage 18′ and the aperture(s) 19′ that open(s) to the passage 18′ may enable at least one condition of a location of a material within which the electrically conductive element 12′ of the dielectric probe 10′ has been inserted to be communicated to a location remote from the electrically conductive element 12′. As an example, the passage 18′ and aperture(s) 19′ may enable a humidity and/or a relative humidity of the material at the location where the dielectric probe 10′ has been inserted to be communicated to a location remote from the location where the dielectric probe 10′ has been inserted. Such communication may occur by way of a conduit 35′ (FIG. 5) that communicates with the passage 18′. Optionally, a coupler 30′ with an orifice 32′ that communicates with the passage 18′ may protrude from the second end 16′ of the electrically conductive element 12′ to facilitate coupling of the conduit 35′ to the electrically conductive element 12′ and the dielectric probe 10′.

Optionally, the dielectric probe 10′ may include a temperature sensor 40′. The temperature sensor 40′ may be carried by the electrically conductive element 12′. For example, the temperature sensor 40′ may be carried by the passage 18′ through the electrically conductive element 12′. As another example, the temperature sensor 40′ may be exposed to an outer surface of the electrically conductive element 12′ (e.g., it may be located within a recess in an outer surface of a wall 13′ of the electrically conductive element 12′, it may be located in an aperture 19′ through the wall 13′ of the electrically conductive element 12′, etc.). In some embodiments, the temperature sensor 40′ may comprise a thermistor. Wires 42′, 44′ may extend from the temperature sensor 40′, along the passage 18′ or another passage through the electrically conductive element 12′ (e.g., a passage formed in the wall 13′, etc.), and through and beyond the second end 16′ of the electrically conductive element 12′.

Another embodiment of a dielectric probe 10″ is illustrated by FIGS. 3, 3A, and 3B. The dielectric probe 10″ includes an electrically conductive housing 12″, a wire 20″, a temperature sensor 40″ with wires 42″ and 44″, and a humidity sensor 50″ with wires 52″ and 54″.

The electrically conductive housing 12″ may be elongated. The electrically conductive housing 12″ may be substantially straight or straight. The electrically conductive housing 12″ includes a sidewall 13″. The sidewall 13″ of the electrically conductive housing 12″ may comprise, consist of, or consist essentially of an electrically conductive material. As an example, the electrically conductive material may be copper, aluminum, steel, or any other suitable electrically conductive material.

The electrically conductive housing 12″ may have any suitable dimensions. For example, the electrically conductive housing 12″ may have a diameter of about three-eighths inch (about 0.95 cm), although other diameters (e.g., 0.4 inch (1 cm)., 0.5 inch (1.25 cm), 0.6 inch (1.5 cm), 0.625 inch (1.6 cm), 0.7 inch (1.8 cm), 0.75 inch (1.9 cm), 1 inch (2.5 cm), etc.) are also within the scope of this disclosure. A length of the electrically conductive housing 12″ may enable it to be embedded vertically a desired distance within a material whose dielectric constant is to be measured. For example, the electrically conductive housing 12″ may have a length of about three inches (about 7.5 cm) or more (e.g., 31/4 inches (8.25 cm), 31/2 inches (8.9 cm), 33/4 inches (9.5 cm), 4 inches (10.1 cm), 41/2 inches (11.4 cm), 5 inches (12.7 cm), 51/2 inches (14 cm), 6 inches (15.25 cm), etc.).

The sidewall 13″ defines at least a portion of an interior 18″ of the electrically conductive housing 12″. One or more apertures 19″ may extend through the sidewall 13″ to establish communication between an exterior of the electrically conductive housing 12″ and the interior 18″ of the electrically conductive housing 12″.

In addition to the sidewall 13″, the electrically conductive housing 12″ has a first end 14″ and a second end 16″.

During use of the dielectric probe 10″, the first end 14″ of the electrically conductive housing 12″ is inserted into a material, such as concrete, whose dielectric constant is to be measured. The first end 14″ may be closed. Such a configuration may prevent a material from entering an interior the passage 18″ while the electrically conductive housing 12″ is introduced into the material.

The first end 14″ may have a shape that facilitates its insertion into the material whose dielectric constant is to be measured. For example, the first end 14″ may be rounded, tapered to a point along the longitudinal axis of the electrically conductive housing 12″, tapered to define a wedge shape, etc.

When the dielectric probe 10″ is used, the second end 16″ of the electrically conductive housing 12″ remains outside of the material whose dielectric constant is to be measured.

A first end 24″ of the wire 20″ of the dielectric probe 10″ may be electrically coupled to the second end 16″ of the electrically conductive housing 12″. Electrical coupling between the first end 24″ of the wire 20″ and the second end 14″ of the electrically conductive housing 12″ may be achieved in any suitable manner. Without limitation, the first end 24″ of the wire 20″ may be soldered to the second end 16″ of the electrically conductive housing 12″, a mechanical coupler may secure the first end 24″ of the wire 20″ to the second end 16″ of the electrically conductive housing 12″ and establish electrical communication between them, etc.

The interior 18″ may open to the second end 16″ of the electrically conductive housing 12″. The interior 18″ and the aperture(s) 19″ that open(s) to the interior 18″ may enable at least one condition of a location of a material within which the electrically conductive housing 12″ of the dielectric probe 10″ has been inserted to be communicated to the interior 18″ of the electrically conductive housing 12″.

The temperature sensor 40″ of the dielectric probe 10″ may be carried by the electrically conductive housing 12″. For example, the temperature sensor 40″ may be carried within the interior 18″ of the electrically conductive housing 12″. As another example, the temperature sensor 40″ may be exposed to an outer surface of the electrically conductive housing 12″ (e.g., it may be located within a recess in an outer surface of the sidewall 13″ of the electrically conductive housing 12″, it may be located in an aperture 19″ through the sidewall 13″ of the electrically conductive housing 12″, etc.). In some embodiments, the temperature sensor 40″ may comprise a thermistor. Wires 42″, 44″ may extend from the temperature sensor 40″, along the interior 18″ or a passage through the electrically conductive housing 12″ (e.g., a passage formed in the sidewall 13″, etc.), and through and beyond the second end 16″ of the electrically conductive housing 12″.

The humidity sensor 50″ of the dielectric probe 10″ may also be carried by the electrically conductive housing 12″. For example, the humidity sensor 50″ may be carried within the interior 18″ of the electrically conductive housing 12″. The humidity sensor 50″, which may also be referred to as a “hygrometer,” may comprise any suitable type of humidity sensor that will fit and function within the interior 18″ (e.g., a chilled mirror hygrometer, etc.). Wires 52″, 54″ may extend from the humidity sensor 50″, along the interior 18″ or a passage through the electrically conductive housing 12″ (e.g., a passage formed in the sidewall 13″, etc.), and through and beyond the second end 16″ of the electrically conductive housing 12″.

Turning now to FIGS. 4 and 4A, an embodiment of a monitoring system 100 is shown. The monitoring system 100 monitors a dielectric constant of a material, such as curing concrete C. The dielectric constant of the material, when compared with a known, standard dielectric constant for the material (e.g., the material in a dry state, etc.), may be used to determine a water content of the material.

The monitoring system 100 includes a plurality of dielectric probes 10 and a base station 102. Optionally, the monitoring system 100 may further include a ground penetrating radar (GPR) device 110.

As illustrated, the dielectric probes 10 may comprise any embodiment of dielectric probe of this disclosure. For example, the dielectric probes 10 may comprise the embodiment of dielectric probe 10 shown in and described in reference to FIGS. 1 and 1A, the embodiment of dielectric probe 10′ shown in and described with reference to FIGS. 2 and 2A, the embodiment of dielectric probe 10″ shown in and described with reference to FIGS. 3 and 3A, or any other embodiment of dielectric probe that may be configured and/or function in the manner described herein.

Two or more dielectric probes 10a, 10b, etc., may be positioned at different locations in the material to be evaluated; for example, the curing concrete C. In embodiments where the dielectric probe 10a, 10b, etc., are placed in curing concrete C, each dielectric probe 10a, 10b, etc., may be positioned within the curing concrete C while the curing concrete C is pliable, or in a plastic state. Alternatively, the dielectric probes 10a, 10b, etc., may be placed after the curing concrete C has set, or once the curing concrete C has partially cured. It may even be desirable to place dielectric probes 10a, 10b, etc., into hardened or fully cured concrete C. The placement of each dielectric probe 10a, 10b, etc., in pliable curing concrete C or even recently set curing concrete C may simply include pushing the dielectric probe 10a, 10b, etc., into the curing concrete C. Placement of the dielectric probes 10a, 10b, etc., into set concrete C, hardened concrete C, or fully cured concrete C may require further force, such as the use of a mallet or a hammer.

The probes 10a, 10b, etc., may be placed in the concrete C at locations that are laterally spaced apart from each other. As shown in FIG. 4B, each probe 10a, 10b, etc., may be oriented substantially vertically or vertically and inserted into the concrete C through an upper surface S of the concrete C. The substantially vertical or vertical orientation of each dielectric probe 10a, 10b, etc., may be maintained while placing the dielectric probe 10a, 10b, etc., into the concrete C. The distance a dielectric probe 10a is inserted into the concrete C, or the depth of the dielectric probe 10a in the concrete C may be the same as or different from the distance at least one other dielectric probe 10b is inserted into the concrete C, or the depth of the dielectric probe 10b in the concrete C.

Each dielectric probe 10a, 10b, etc., may communicate with the base station 102. The base station 102 may comprise a concrete curing maturity meter, which may measure a dry bulb temperature (T) and dew point temperature (Dₚ) of the concrete C, from which relative humidity (RH) may be calculated, as well as the temperature (e.g., dry bulb temperature, etc.), relative humidity, wind speed, and solar radiation of the environment in which the curing concrete C is located.

With the dielectric probes 10a, 10b, etc., in place, they may be electrically coupled to the base station 102. For example, a wire 24a of one dielectric probe 10a may be electrically coupled to an output connector 103a of the base station 102, while a wire 24b of another dielectric probe 10b may be electrically coupled to an input connector 103b of the base station 102. The base station 102 may then send electricity (i.e., an electrical current) through the connector 103a and the wire 24a to dielectric probe 10a. Electricity that passes into and through the concrete C may then be received by dielectric probe 10b and be communicated back to the base station 102 through wire 24b and connector 103b. Based on differences between the electricity sent to dielectric probe 10a and the electricity received by dielectric probe 10b, depths the probes 10a and 10b have been inserted into the concrete C, and distances the probes 10a and 10b protrude from the concrete, a processor 105 of the base station 102 may determine a dielectric constant of a location or a region of the concrete C, which may be used to determine a water content of the concrete C.

The GPR device 110 may comprise any suitable device for evaluating an area from above the surface of the area. The GPR device 110 may provide further information on the concrete C. For example, the GPR device 110 may provide information on suction strength of the water within the concrete C, changes in dimensions and/or shape of the concrete C over time, etc. The information obtained by the GPR device 110 may be used with the information obtained by the probes 10 and the base station 102 to evaluate changes in the concrete C or another material over time.

The information obtained with the base station 102 and the optional GPR device 120 to provide information on a condition of the concrete C, determine whether any corrective action should be taken to address the condition of the concrete C, and, optionally, identify the corrective action to be taken to address the condition of the concrete C. For example, the information may be used to determine the extent to which various locations of the curing concrete C are hydrated and hydration/drying trends across the surface of the curing concrete C. That information may then be used to determine how to treat the curing concrete C, if at all, such as aby further hydrating one or more locations of the curing concrete C and/or applying curing aids to one or more locations of the curing concrete C.

FIG. 5 depicts an embodiment of a monitoring system 100′ that includes a base station 102′ and a plurality of dielectric probes 10′ that facilitate measurement of the dielectric constant of a portion of a material, such as curing concrete C, and measurement of a humidity of the material. The monitoring system 100′ may optionally include a GPR device 110′.

Two or more dielectric probes 10a′, 10b′, etc., may be positioned at different locations in the material to be evaluated; for example, the curing concrete C. In embodiments where the dielectric probe 10a′, 10b′, etc., are placed in curing concrete C, each dielectric probe 10a′, 10b′, etc., may be positioned within the curing concrete C while the curing concrete C is pliable, or in a plastic state. Alternatively, the dielectric probes 10a′, 10b′, etc., may be placed after the curing concrete C has set, or once the curing concrete C has partially cured. It may even be desirable to place dielectric probes 10a′, 10b′, etc., into hardened or fully cured concrete C. The placement of each dielectric probe 10a′, 10b′, etc., in pliable curing concrete C or even recently set curing concrete C may simply include pushing the dielectric probe 10a′, 10b′, etc., into the curing concrete C. Placement of the dielectric probes 10a′, 10b′, etc., into set concrete C, hardened concrete C, or fully cured concrete C may require further force, such as the use of a mallet or a hammer.

The probes 10a′, 10b′, etc., may be placed in the concrete C at locations that are laterally spaced apart from each other. Each probe 10a′, 10b′, etc., may be oriented substantially vertically or vertically and inserted into the concrete C through an upper surface S of the concrete C. The substantially vertical or vertical orientation of each dielectric probe 10a′, 10b′, etc., may be maintained while placing the dielectric probe 10a′, 10b′, etc., into the concrete C. The distance a dielectric probe 10a′ is inserted into the concrete C, or the depth of the dielectric probe 10a′ in the concrete C may be the same as or different from the distance at least one other dielectric probe 10b′ is inserted into the concrete C, or the depth of the dielectric probe 10b′ in the concrete C.

The base station 102′ may comprise a concrete curing maturity meter, which may measure a dry bulb temperature (T) and dew point temperature (Dₚ) of the concrete C, from which relative humidity (RH) may be calculated, as well as the temperature (e.g., dry bulb temperature, etc.), relative humidity, wind speed, and solar radiation of the environment in which the curing concrete C is located.

With the dielectric probes 10a′, 10b′, etc., in place, they may be electrically coupled to the base station 102. For example, a wire 24a′ of one dielectric probe 10a′ may be electrically coupled to an output connector 103a′ of the base station 102′, while a wire 24b′ of another dielectric probe 10b′ may be electrically coupled to an input connector 103b′ of the base station 102′. The base station 102′ may then send electricity through the connector 103a′ and the wire 24a′ to dielectric probe 10a′. Electricity that passes into and through the concrete C may then be received by dielectric probe 10b′ and be communicated back to the base station 102′ through wire 24b′ and connector 103b′. Based on differences between the electricity sent to dielectric probe 10a′ and the electricity received by dielectric probe 10b′, depths the probes 10a′ and 10b′ have been inserted into the concrete C, and lengths of the probes 10a′ and 10b′ that protrude from the concrete, a processor 105′ of the base station 102′ may determine a dielectric constant of a location or a region of the concrete C.

In addition, each dielectric probe 10a′, 10b′, etc., may be fluidly coupled to the base station 102′. More specifically, a conduit 35′ may fluidly couple a passage 18′ (FIGS. 2A, and 2B) through each dielectric probe 10a′, 10b′, etc., with a fluid coupler 105′ of the base station 102′. The conduit 35′ and the coupler 105′ may communicate humidity collected by the passage 18′ of each dielectric probe 10a′, 10b′, etc., to a humidity sensor 106′ (e.g., a chilled mirror hygrometer, etc.) of the base station 102′.

The dielectric constant of the concrete C, the water content of the concrete C, as determined from the dielectric constant, and the humidity of the concrete C may then be used with other information (e.g., dry bulb temperature, relative humidity, etc.), to provide information on a condition of the concrete C, determine whether any corrective action should be taken to address the condition of the concrete C, and, optionally, identify the corrective action to be taken to address the condition of the concrete C.

The optional GPR device 110′ may comprise any suitable device for evaluating an area from above the surface of the area. The GPR device 110′ may provide further information on the concrete C. For example, the GPR device 104 may provide information on suction strength of the water within the concrete C, changes in dimensions and/or shape of the concrete C over time, etc. The information obtained by the GPR device 110′ may be used with the information obtained by the probes 10 and the base station 102′ to evaluate changes in the concrete C or another material over time. For example, the information may be used to determine the extent to which various locations of the curing concrete C are hydrated and hydration/drying trends across the surface of the curing concrete C. That information may then be used to determine how to treat the curing concrete C, if at all, such as aby further hydrating one or more locations of the curing concrete C and/or applying curing aids to one or more locations of the curing concrete C.

Although the preceding disclosure provides many specifics, these should not be construed as limiting the scope of any of the claims that follow, but merely as providing illustrations of some embodiments of elements and features of the disclosed subject matter. Other embodiments of the disclosed subject matter may be devised which do not depart from the spirit or scope of any of the claims. Features from different embodiments may be employed in combination. Accordingly, the scope of each claim is limited only by its plain language and the legal equivalents thereto.

What is claimed:

1. A dielectric probe for use in concrete, comprising:
an electrically conductive element including a tip that facilitates insertion of the body into the concrete; and
a wire extending from the electrically conductive element to facilitate measurement of a dielectric constant of the concrete with the electrically conductive element.

2. The dielectric probe of claim 1, wherein the electrically conductive element comprises a solid elongated structure formed from an electrically conductive material.

3. The dielectric probe of claim 1, wherein the electrically conductive element comprises a sample tube with a passage extending partially therethrough and at least one aperture extending between the passage and an exterior of a sidewall of the electrically conductive element.

4. The dielectric probe of claim 3, wherein the passage communicates humidity from the concrete to a humidity sensor remote from the dielectric probe.

5. The dielectric probe of claim 1, wherein an interior is defined within the electrically conductive element and at least one aperture establishes communication between the interior and an exterior of a sidewall of the electrically conductive element.

6. The dielectric probe of claim 5, further comprising:
a relative humidity sensor carried within the interior.

7. A monitoring system for curing concrete, comprising:
a plurality of dielectric probes; and
a base station.

8. The monitoring system of claim 7, wherein the plurality of dielectric probes comprises a pair of dielectric probes, including:
a first dielectric probe that receives electricity from the base station and coveys the electricity into the curing concrete; and
a second dielectric probe that receives the electricity from the curing concrete and conveys the electricity back to the base station.

9. The monitoring system of claim 8, wherein the base station includes a processor programmed to calculate a dielectric constant of the curing concrete based on a change in the electricity.

10. The monitoring system of claim 9, wherein the processor is further programmed to calculate a water content of the concrete based on the dielectric constant of the curing concrete.

11. The monitoring system of claim 8, wherein the first dielectric probe and the second dielectric probe include elongated electrically conductive elements positionable within the curing concrete vertically and in spaced-apart relation to each other.

12. A method for characterizing a change in measured strain of curing concrete, comprising:
introducing a pair of dielectric probes into the curing concrete through a surface of the curing concrete at spaced apart locations and parallel orientations, a dielectric constant of the curing concrete measureable with the pair of dielectric probes;
measuring the dielectric constant of the curing concrete between the pair of dielectric probes;
determining a water content of the curing concrete at a location between the dielectric probes; and
determining the measured strain of the curing concrete at the location between the pair of dielectric probes as a function of at least the dielectric constant of the curing concrete.

13. The method of claim 12, further comprising:
measuring a humidity of the curing concrete at the locations of the pair of dielectric probes; and
determining the measured strain of the curing concrete based at least partially on the humidity of the concrete.

14. The method of claim 12, wherein introducing the pair of dielectric probes comprises forcing tips of electrically conductive elements of the pair of dielectric probes into and through the surface of the curing concrete.

15. The method of claim 12, wherein introducing the pair of dielectric probes comprises introducing a plurality of dielectric probes into the different locations of the surface spaced apart from each other substantially evenly over the surface.

16. The method of claim 12, wherein introducing the pair of dielectric probes comprises introducing the pair of dielectric probes into pliable concrete.

17. The method of claim 12, wherein introducing the pair of dielectric probes comprises introducing the pair of dielectric probes into set concrete.

18. The method of claim 12, wherein introducing the pair of dielectric probes comprises introducing the pair of dielectric probes into curing concrete that has been in place at least seven days.

19. The method of claim 12, further comprising:
treating the curing concrete in response to the measured strain.
